(19) United States
(12) Patent Application Publication
(10) Pub. No.: US 2022/0387477 A1
(43) Pub. Date: Dec. 8, 2022

(54) PHARMACEUTICAL COMPOSITIONS CONTAINING ALGINATE OLIGOSACCHARAIDE DIACID

(71) Applicant: GREEN VALLEY (SHANGHAI) PHARMACEUTICALS CO., LTD, Shanghai (CN)

(72) Inventors: Yingshen Jin, Shanghai (CN); Xianliang Xin, Shanghai (CN); Zhongping Xiao, Shanghai (CN); Zhenqing Zhang, Shanghai (CN); Meiyu Geng, Shanghai (CN)

(21) Appl. No.: 17/772,775

(22) PCT Filed: Oct. 26, 2020
(86) PCT No.: PCT/CN2020/123750

§ 371 Date: Apr. 28, 2022

(30) Foreign Application Priority Data
Oct. 31, 2019 (CN) 201911057518.8

(51) Int. Cl. A61K 31/734; A61K 9/20

(52) U.S. Cl. CPC A61K 31/734; A61K 9/2059; A61K 9/2054; A61K 9/2095

(57) ABSTRACT

The invention relates to a pharmaceutical composition containing alginate oligosaccharide diacid with formula (IV) or a pharmaceutically acceptable salt thereof, which can be used for preparing medicaments for treating Alzheimer's disease, Parkinson's disease, inflammation, pain, diabetes or vascular dementia.

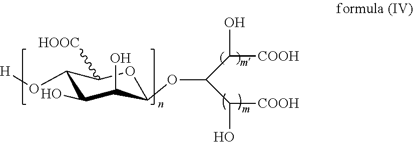

### TECHNICAL FIELD

The present invention relates to a pharmaceutical composition prepared by taking alginate oligosaccharide diacid or a pharmaceutically acceptable salt thereof as raw materials and a preparation method thereof; and belongs to the technical field of medicine.

### BACKGROUND

Alginic oligosaccharides have been paid extensive attention due to their potential medicinal values. Alginic oligosaccharides are usually prepared by multiple steps with alginic acid as a raw material.

In the alginic oligosaccharide molecules, there is an M segment formed of D-mannuronic acids linked by β-1,4-glucosidic bonds, a G segment formed of L-guluronic acids linked by α-1,4-glucosidic bonds, and an MG segment formed by hybridization of the two saccharides. The structural formulae of D-mannuronic acid and L-guluronic acid are shown in Formula (I) and Formula (II) below: the algin oligosaccharide has a structural formula shown as the following formula (III):

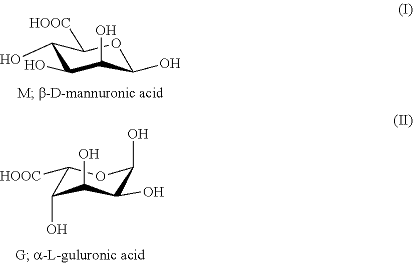

The structural formula of alginic oligosaccharides is shown by Formula (III) below:

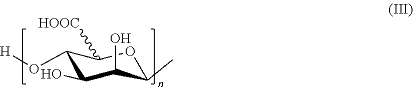

The prior patent application CN201711467596.6 discloses a mannuronic acid oligosaccharide combination comprising mannuronic acid with the formula (V) or a pharmaceutically acceptable salt thereof:

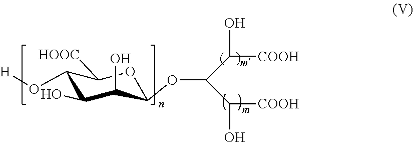

wherein n is an integer selected from 1 to 9, m is selected from 0, 1 or 2, m′ is selected from 0 or 1,

and wherein,

the total weight of mannuronic acid wherein n=1-5 accounts for 80-95% of the total weight of the combination;

the ratio of the total weight of the mannuronic diacids wherein n=1-3 to the total weight of the mannuronic diacids wherein n=4-7 is between 1.0 and 3.5.

The mannuronic diacid oligosaccharide combination can inhibit cell damage, protect nerve cells, and increase cell survival rate. It has potential effects of preventing and treating Alzheimer disease.

In order to obtain the oligomannaric acid which has effects against Alzheimer's disease (AD) and Diabetes Mellitus in the previous patent, guluronic acid in the raw material alginic acid is removed, and the content of guluronic acid in alginic acid is usually above 30% and up to about 70%, so the actual production cost is very high in order to obtain high-purity oligomannaric acid. Therefore, the development of pharmaceutical compositions of alginate oligosaccharide diacid suitable for use meets the urgent need of the industry.

### DISCLOSURE OF INVENTION

One aspect of the invention provides a pharmaceutical composition, comprising 40-90 wt % of alginate oligosaccharide diacid or a pharmaceutically acceptable salt thereof, the structure of which is shown in formula (IV), and 8-50 wt % of a filling agent; wherein the filler is selected from starch and/or cellulose;

wherein n is an integer selected from 1 to 9, m is selected from 0, 1 or 2, and m′ is selected from 0 or 1,

and wherein,

the total weight of the alginate oligosaccharide diacids wherein n=1-5 accounts for more than 60% of the total weight of the combination; the total weight of guluronic acid diacid is less than 50% of the weight of the composition.

the alginic oligosaccharic acid composition of the present invention is a mixture of mannuronic acid and guluronic acid with different polymerization degrees, and its main components are oligosaccharide with a polymerization degree of 2 to 10: an M segment formed of mannuronic acids linked by β-1,4-glucosidic bonds, a G segment formed of guluronic acids linked by α-1,4-glucosidic bonds, and an MG segment formed by hybridization of the two saccharides. It is known that mannuronic diacids have certain pharmacological activities against Alzheimer's disease (AD) and Diabetes Mellitus. The most active saccharides are pentasaccharide to octasaccharide, especially hexasaccharide. However, the inventors find that the oligosaccharic diacid mixture of mannuronic acid and guluronic acid with a polymerization degree of 2 to 10 also has pharmacological activities against Alzheimer's disease (AD) and Diabetes Mellitus, but the premise is that the content of guluronic acid is controlled within a certain range. In other words, the alginic oligosaccharic diacid composition of the present invention can be prepared with greatly reduced production cost, which makes it easier to realize in actual production and easy to realize industrialized mass production.

In addition, alginate oligosaccharide diacid is very hygroscopic and can be largely decomposed under the conditions of high temperature, strong alkali, oxidation etc. Meanwhile, since mannuronic acid and guluronic acid may be degraded during the treatment process, formic acid impurities which are harmful to the human body are generated, and thus quality control of raw materials is required to obtain an oral preparation having high safety and effectiveness. The inventors of the present application have studied and developed a novel pharmaceutical composition of alginate oligosaccharide diacid that is more stable and can be rapidly dissolved

The purity of the alginate oligosaccharide diacid or a pharmaceutically acceptable salt thereof used in the present invention is greater than 90%, preferably greater than 95%, more preferably greater than 98%. The sources of impurities are complex, and the applicant finds that one or more of the following substances may be generated in the preparation process of the alginate oligosaccharide diacid:formic acid, glycolic acid, oxalic acid, malonic acid, hydroxymalonic acid, tartaric acid, etc. Without being bound by any theory, and without limiting the types of the impurities of the present invention, these impurities may originate from the oxidation step, or from impurities in the initial algal feedstock that are not readily detectable or from the processing of the initial algal feedstock. The inventors found that formic acid, among these impurities, is more difficult to be removed due to its small molecular weight, and has a significant influence on the stability of the formulation. Therefore, controlling the content of formic acid of the alginate oligosaccharide diacid or a pharmaceutically acceptable salt thereof is one of the important aspects of the present invention. Without being bound by any theory, controlling the particle size of the active ingredient in the formulation within a certain range results in a more stable formulation with less formic acid impurity.

Another aspect of the invention relates to the use of a pharmaceutical composition in the preparation of a medicament for the treatment of a disease selected from the group consisting of Alzheimer's disease, Parkinson's disease, inflammation, pain, diabetes and vascular dementia.

Yet another aspect of the invention relates to a method of treating a patient suffering from a disease selected from the group consisting of: Alzheimer's disease, Parkinson's disease, inflammation, pain, diabetes or vascular dementia, comprising administering to a patient in need thereof a pharmaceutical composition according to the invention in unit dosage form.

Yet another aspect of the invention relates to a pharmaceutical formulation comprising a pharmaceutical composition according to the invention.

Another aspect of the present invention relates to a method for preparing the pharmaceutical composition of the present invention, comprising the steps of:

(1) Mixing 40-90 wt % of alginate oligosaccharide diacid or a pharmaceutically acceptable salt thereof and 8-50 wt % of filler uniformly;

(2) The uniformly mixed material is granulated, and packed into a packaging bag quantitatively, or filled into a capsule, or tableted to obtain the pharmaceutical composition; or the uniformly mixed material obtained in step (1) is directly tableted to obtain the described pharmaceutical composition.

The formulation of the alginate oligosaccharide diacid or the salt thereof has excellent preparation stability, can be quickly dissolved in an in vivo environment, and can ensure that the active ingredients of the alginate oligosaccharide diacid can be quickly absorbed by a human body. In addition, the inventor also solved the problems arised from the preparation process of high-load alginate oligosaccharide diacid through the composition design of the preparation, including the uniformity of the formulation, the uniform particle distribution etc.

### DRAWINGS

FIG. 1 shows the NMR spectrum of the intermediate.

FIG. 2 shows the mass spectrum of disaccharide, trisaccharide and tetrasaccharide in alginate-oligosaccharide diacid API.

FIG. 3 shows a mass spectrum of pentasaccharide, hexasaccharide and heptasaccharide in alginate oligosaccharide diacid API.

FIG. 4 shows a mass spectrum of octaose, nonaose and decaose in alginate oligosaccharide diacid API.

FIG. 5 shows the NMR spectra of the alginate oligosaccharide diacid API.

### DETAILED DESCRIPTION

Various aspects of the present invention will be described in detail below, but the present invention is not limited to these specific embodiments. Modifications and adaptations of the present invention that come within the spirit of the following disclosure may be made by those skilled in the art and are within the scope of the present invention.

The invention provides a pharmaceutical composition, which comprises 40-90 wt % of alginate oligosaccharide diacid or a pharmaceutically acceptable salt thereof, wherein the structure of the alginate oligosaccharide diacid is shown in a formula (IV), and 8-50 wt % of the filler; wherein the filler is at least one selected from starch and cellulose;

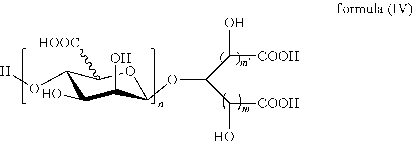

wherein n is an integer selected from 1 to 9, m is selected from 0, 1 or 2, and m′ is selected from 0 or 1.

Preferably, the filler is selected from at least one of the group consisting of dry starch, corn starch, pregelatinized starch, microcrystalline cellulose, sodium carboxymethylcellulose, and croscarmellose sodium.

Preferably, the pharmaceutical composition of the present invention further comprises 0 to 10 wt % of a binder and 0.1 to 3 wt % of a lubricant; wherein the binder is at least one of starch slurry, sodium carboxymethylcellulose, hydroxypropyl cellulose, hydroxypropyl methylcellulose and polyvinylpyrrolidone; the lubricant is at least one selected from talc, magnesium stearate, silica gel micropowder, and hydrogenated vegetable oil.

Preferably, the pharmaceutical composition of the present invention comprises 60-75 wt % of alginate oligosaccharide diacid or a pharmaceutically acceptable salt thereof, 25-35 wt % of filler and 1-3 wt % of lubricant.

Preferably, the pharmaceutical composition of the present invention comprises 80-90 wt % of alginate oligosaccharide diacid or a pharmaceutically acceptable salt thereof, 8-16 wt % of filler and 1-3 wt % of lubricant.

Preferably, the particle size of the alginate oligosaccharide diacid or a pharmaceutically acceptable salt thereof (API) used in the pharmaceutical composition of the present invention is controlled such that 100 wt % pass through a 60 mesh sieve, and that more than 70 wt % pass through a 100 mesh sieve. Preferably, more than 80 wt % pass through a 100 mesh sieve, more preferably, more than 90 wt % pass through a 100 mesh sieve. Since the active ingredient has the characteristics of being easily soluble and hygroscopic, in order to make the active pharmaceutical ingredient play a better role, the inventor found that, controlling the ratio of the API particle size below 100 mesh powder and appropriately reducing the coarse raw material larger than 60 mesh will help to improve the dissolution rate of capsules and the shape of intermediate particles after granulation, respectively.

Preferably, in the pharmaceutical composition of the present invention, the total weight of the alginate oligosaccharide diacid wherein n=1 to 5 accounts for more than 60% of the total weight of the alginate oligosaccharide diacid or a pharmaceutically acceptable salt thereof; the total weight of guluronic acid diacid accounts for 0-50% of the total weight of alginate oligosaccharide diacid or a pharmaceutically acceptable salt thereof.

Preferably, in the pharmaceutical composition of the present invention, the total weight of mannuronic acid and/or guluronic acid wherein n=1 to 3 accounts for 20 to 70% of the total weight of alginate oligosaccharide diacid or a pharmaceutically acceptable salt thereof.

Preferably, in the pharmaceutical composition of the present invention, the total weight of guluronic acid diacid accounts for 5-50%, preferably 10-40%, more preferably 15-35% of the total weight of alginate oligosaccharide diacid or a pharmaceutically acceptable salt thereof.

Preferably, in the pharmaceutical composition of the present invention, the weight of the alginate oligosaccharide diacid or a pharmaceutically acceptable salt thereof with each polymerization degree accounts for the total weight of the alginate oligosaccharide diacid or a pharmaceutically acceptable salt thereof in percentage by weight as follows: 5-30% of disaccharide (n=1), 15-35% of trisaccharide (n=2), 15-35% of tetrasaccharide (n=3), 20-45% of the total content of pentasaccharide (n=4), hexasaccharide (n=5), heptasaccharide (n=6), octasaccharide (n=7), nonasaccharide (n=8) and decasaccharide (n=9).

Preferably, in the pharmaceutical composition of the present invention, the weight of the alginate oligosaccharide diacid or a pharmaceutically acceptable salt thereof with each polymerization degree accounts for the total weight of the alginate oligosaccharide diacid or a pharmaceutically acceptable salt thereof in percentage by weight as follows: 5-30% of disaccharide, 15-35% of trisaccharide, 15-35% of tetrasaccharide, 10-25% of pentasaccharide, 5-15% of hexasaccharide, 3-10% of heptasaccharide, 2-5% of octasaccharide, 1-5% of nonasaccharide and 1-5% of decasaccharide.

Preferably, in the pharmaceutical composition of the present invention, the weight of the alginate oligosaccharide diacid or a pharmaceutically acceptable salt thereof with each polymerization degree accounts for the total weight of the alginate oligosaccharide diacid or a pharmaceutically acceptable salt thereof in percentage by weight as follows: 10-29% of disaccharide, 18-32% of trisaccharide, 15-30% of tetrasaccharide, 15-20% of pentasaccharide, 5-10% of hexasaccharide, 3-5% of heptasaccharide, 2-3% of octasaccharide, 1-3% of nonasaccharide and 1-3% of decasaccharide.

In a preferred embodiment, the pharmaceutical composition according to any of the preceding claims, characterized in that the content of formic acid or a salt thereof is below 0.2%, preferably below 0.1%, more preferably below 0.05%.

Preferably, the pharmaceutical composition of the present invention comprises sodium or potassium alginate oligosaccharide diacid salt as a pharmaceutically acceptable salt.

Preferably, the pharmaceutical composition of the present invention comprises sodium alginate oligosaccharide diacid salt as a pharmaceutically acceptable salt in an amount of about 100 mg to 500 mg, preferably 150 mg to 450 mg.

Another aspect of the invention relates to the use of a pharmaceutical composition for the preparation of a medicament for the treatment of a condition selected from the group consisting of Alzheimer's disease, Parkinson's disease, inflammation, pain, diabetes and vascular dementia.

Chinese patent application CN201810721327.6 discloses that oligosaccharide diacid mixture of mannuronic acid and guluronic acid (the alginate oligosaccharide diacid API described in the present invention) proved to have pharmacological activity against Alzheimer's disease, Parkinson's disease, inflammation, pain, diabetes or vascular dementia. Therefore, the pharmaceutical composition containing alginate derived oligosaccharide diacid or the salt thereof also has pharmacological activity against Alzheimer's disease, Parkinson's disease, inflammation, pain, diabetes or vascular dementia.

Pain mentioned in the present invention includes various pains, including but not limited to acute pain, chronic pain, neuropathic pain, postoperative pain, chronic low back pain, cluster headache, herpes neuralgia, phantom limb pain, central pain, toothache, opioid-resistant pain, visceral pain, surgical pain, bone injury pain, fatigue and pain during childbirth, pain caused by burns including sunburn, postpartum pain, migraine, angina, and genitourinary tract related pain (including cystitis), vascular pain, trigeminal neuralgia, intercostal neuralgia, surgical incision pain, chronic fasciitis pain, heel pain, muscle pain, bone pain, joint pain, cancer pain, non-cancerous pain etc.

Inflammation mentioned in the present invention includes various inflammations, including but not limited to acute inflammation, chronic inflammation, vascular inflammation, neuroinflammation, central nervous system inflammation (such as multiple sclerosis, including encephalomyelitis, etc.), peripheral nerve inflammation, Arthritis (such as osteoarthritis, sacroiliitis, etc., psoriatic arthritis, rheumatoid arthritis, rheumatoid arthritis, etc.), ankylosing spondylitis, inflammatory bowel disease (such as Crohn's disease and ulcerative colon inflammation), inflammatory diabetic ulcers, systemic lupus erythematosus, inflammatory skin diseases (such as psoriasis, atopic dermatitis, eczema), etc.

Another aspect of the invention relates to a method of treating a patient suffering from a disease selected from the group consisting of Alzheimer's disease, parkinson's disease, inflammation, pain, diabetes or vascular dementia, comprising administering to a patient in need thereof a pharmaceutical composition according to the invention in unit dosage form.

Preferably, the unit dose contains 100 mg to 500 mg alginate oligosaccharide diacid sodium salt, preferably 150 mg to 450 mg. The unit dose may be administered to the patient once or more daily.

Another aspect of the invention relates to a pharmaceutical formulation comprising the pharmaceutical composition according to the invention.

Preferably, the pharmaceutical composition of the present invention is suitable for oral administration. The use of the pharmaceutical compositions according to the invention and preferred oral dosage forms are tablets, granules, capsules, lozenges, coated tablets and other solid oral dosage forms.

Another aspect of the present invention relates to a method for preparing the pharmaceutical composition of the present invention, comprising the steps of:

(1) Mixing 40-90 wt % of alginate derived oligosaccharide diacid or a pharmaceutically acceptable salt thereof and 8-50 wt % of filler uniformly;

(2) The uniformly mixed material is granulated, and packed into a packaging bag in a quantitative manner, or filled into a capsule, or tableted to obtain the pharmaceutical composition; or directly tableting the uniformly mixed material obtained in step (1) to obtain the pharmaceutical composition.

Preferably, 0 to 10 wt % of a binder, preferably 1 to 10 wt %, more preferably 2 to 8 wt % may also be added during the preparation. It can be added when the filler and the alginate oligosaccharide diacid or a pharmaceutically acceptable salt thereof in the step (1) are mixed; it can also be added during the granulation in the step (2). The binder is at least one selected from the group consisting of starch slurry, sodium carboxymethylcellulose, hydroxypropyl cellulose, hydroxypropyl methylcellulose, and polyvinylpyrrolidone. Alternatively, the binder may be an aqueous solution of a lower alkanol, such as methanol, ethanol, or isopropanol.

Preferably, in the preparation method of the present invention, the granulation in step (2) is dry granulation, wet granulation or boiling granulation.

Preferably, in the preparation method of the present invention, 0.1 to 3 wt % of lubricant may be further added after the completion of the granulation in step (2).

Preferably, in the preparation method of the present invention, step (2) is followed by step (3): coating the plain tablets obtained by tableting in the step (2) to obtain the pharmaceutical composition.

Preferably, the preparation method of the invention comprises the following steps:

(1) Mixing 40-90 wt % of alginate oligosaccharide diacid or a pharmaceutically acceptable salt thereof and 8-50 wt % of filler uniformly;

(2) Adding binder to the uniformly mixed material to make soft material, granulating with a sieve with an aperture of 4±0.5 mm, drying at 60-70° C., and granulating with a mesh with an aperture of 1.2±0.2 mm, adding lubricant, mixing uniformly, and making into capsule or tablet to thin film coat.

The pharmaceutical composition of the present invention selects a specific filler, binder or lubricant, and is combined with alginate oligosaccharide or a pharmaceutically acceptable salt thereof in a specific ratio; the composition has good stability and high disintegration and dissolution rate. For example, the dissolution rate can reach more than 90% in 30 minutes.

Advantages of the present invention are further illustrated in the following non-limiting examples. However, the specific materials and amounts thereof as well as other experimental conditions used in the examples should not be construed as limiting to the present invention. Unless otherwise specified, the parts, proportions, percentages, and the like in the present invention are all calculated by mass.

### EXAMPLES

The measurement methods employed in the following specific examples are as follows.

Dissolution Rate Detection Method: Carbazole Sulfate Method

According to the determination method of dissolution rate and release method (0931 the first method in the fourth general rule of the “Chinese pharmacopoeia” 2015 edition), 900 mL of acetic acid-sodium acetate buffer solution (pH4.5) was used as a dissolution medium, the rotation speed is 50 r/min, the method was operated according to the method. After 30 min, a proper amount of solution was taken out. According to carbazole sulfate method, sodium glucuronate was used as a standard substance to make a standard curve, and the content of dissolution liquid and a control solution were calculated to obtain the dissolution rate.

The Determination Method of the Content of Formic Acid:

Referring to 0521 Gas Chromatography in Chinese Pharmacopoeia, “General Principles”, 2015 edition.

The Determination Method of Weight Average Molecular Weight and Molecular Weight Distribution:

Size Exclusion Chromatography-Light Scattering System

Chromatographic conditions: waters SEC chromatography column (Waters ACQUITY UPLC@BEH125 SEC 1.7 μm 4.6×300 mm column, column temperature 25° C.; the mobile phase was methanol: 80 mmol/L ammonium acetate 1:4 (V/V); the flow rate was 0.1 mL/min; detector: an eighteen angle laser detector (LS) and a refractive index detector (RI, 35° C.).

The sample to be detected was diluted with a mobile phase to prepare a solution which is 10 mg/mL of alginate oligosaccharide as a test solution; 20 μl of test solution was injected into a liquid chromatogram, and was analyzed to obtain a multi-angle laser scattering-differential refraction chromatogram spectrum; the data was processed by spectrum processing software (ASTRA) and the weight average molecular weight and the molecular weight distribution of the sample were obtained.

### Example 1: Preparation of Alginate Oligosaccharide Diacid or a Pharmaceutically Acceptable Salt Thereof (API)

Step 1): Preparation of an Alginic Acid Oligosaccharide Mixture

5 Kg of sodium alginicate was prepared into a solution of about 10%, and the pH was adjusted to about 3.0 by adding dilute hydrochloric acid. The solution was heated to 80° C., and stirred. It was allowed to react for 10 hr before the heating was stopped. After cooling to room temperature, the pH was adjusted to 9.0 by adding NaOH, and further adjusted to 3.2 by adding dilute hydrochloric acid. The solution was centrifuged at 5000 rpm for 10 min. The supernatant was collected, and adjusted to pH 1.0 by adding HCl. After centrifugation, the precipitate was collected, concentrated on a rotary evaporator, and dried under vacuum to give 1500 g of the intermediate.

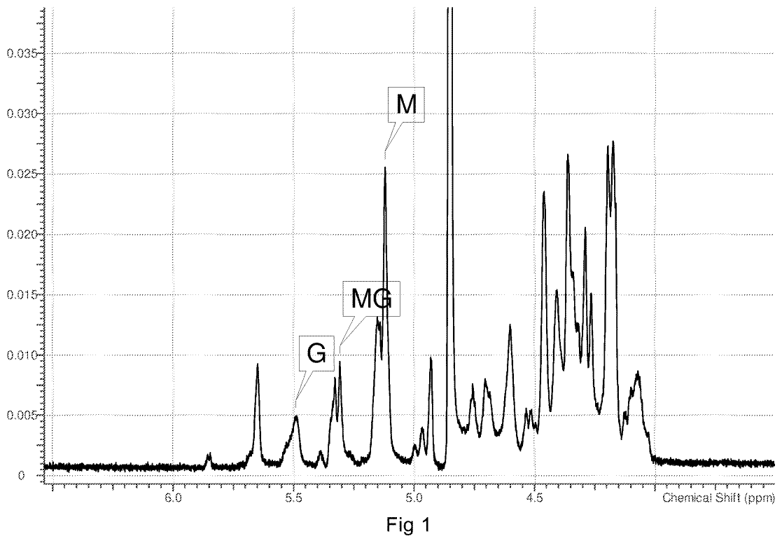

See FIG. 1 for the NMR spectrum of the intermediate. The NMR analysis was conducted as follows: sample preparation: 30 mg of the sample to be tested was weighed and dissolved in 0.5 ml D2O, and lyophilized; 0.5 ml deuterated heavy water was further added for dissolution; lyophilization was performed again; and finally, the lyophilized sample powder was dissolved with an appropriate amount of heavy water, transferred to an NMR tube, and prepared to a 100 mg/ml solution to be tested; 0.01% (w/v) deuterated TSP (trimethylsilylpropionic) sodium salt was added as an internal standard. Nuclear magnetic data acquisition and processing: 400M Fourier transform nuclear magnetic resonance instrument collected one-dimensional hydrogen spectrum at 60° C. The pulse sequence was 45° pulses, each acquisition was 4 seconds, the relaxation time was 1 second, and the accumulation was 20 times, and the spectral width was from −2 ppm to 10 ppm. After data collection, Fourier transform was used to obtain a one-dimensional hydrogen spectrum, and the TSP methyl hydrogen signal was set to 0.00 ppm.

It can be seen from FIG. 1 that the intermediate contained a mannuronic acid segment (M-block, chemical shift 5.1 ppm) and a guluronic acid segment (G-block, chemical shift 5.5 ppm), as well as a chimeric segment of mannuronic acid and guluronic acid (MG-block, chemical shift 5.3 ppm). 500 g of the intermediate was weighed, and dissolved in distilled water to prepare a solution in a volume of 5 L. The solution was adjusted to pH 6.5 with NaOH, and heated in a water bath to control the reaction temperature at 75° C. The gas flow rate at the outlet of an oxygen cylinder and the power of an ozone generator were adjusted such that ozone was fed into the reaction solution at a mass concentration flow rate of 8 g/hr. After 4 hr of reaction, the feeding of ozone was stopped, and a suitable amount of water was added to adjust the concentration of the solution to about 10%. The solution was filtered through an ultrafiltration membrane with a molecular weight cut-off of 2,000 Da to collect a retentate. The collected liquid was concentrated on a rotary evaporator and dried under vacuum to obtain 350 g API of alginate oligosaccharide diacid.

Step 2): Analysis of Proportions and Structures of Oligosaccharides with Various Polymerization Degrees in Alginic Oligosaccharic Diacid API

100 mg of the above dried alginic oligosaccharic diacid API was accurately weighed, dissolved in water to a concentration of 10 mg/mL, and passed through a 0.22 um filter membrane to obtain a test sample solution. The proportions of oligosaccharides with different polymerization degrees in the composition were determined by Superdex peptide molecular exclusion chromatography (GE Co.) in combination with multi-angle laser light scattering (MALS, Wyatt Co.). The experimental conditions were as follows:

Chromatographic column: Superdex peptide 10/300G1

Mobile phase: 0.1 mol/L NaCl

Injection volume: 10 μL

Flow rate: 0.3 mL/min

Test results: from disaccharide to decasaccharide were represented by dp2-dp10, respectively, dp2 was 18%, dp3 was 24%, dp4 was 23%, dp5 was 14%, dp6 was 8%, dp7 was 7%, dp8 was 2%, dp9 was 2% and dp10 was 2%.

Step 3): LC-MS Analysis of Structures of Oligosaccharides with Various Polymerization Degrees in Alginic Oligosaccharic Diacid API

Experimental Conditions:

Chromatographic column: Superdex peptide 10/300G1

Mobile phase: 20% methanol+80% 80 mmol/L NH₄Ac

Flow rate: 0.1 mL/min

Column temperature: 25° C.±0.8° C.

Mass spectrometry conditions: Agilent 6540 QTOF; ion source:

ESI collision voltage 120 V; negative ion mode. The width of the acquired signal (m/z) was 100-1000.

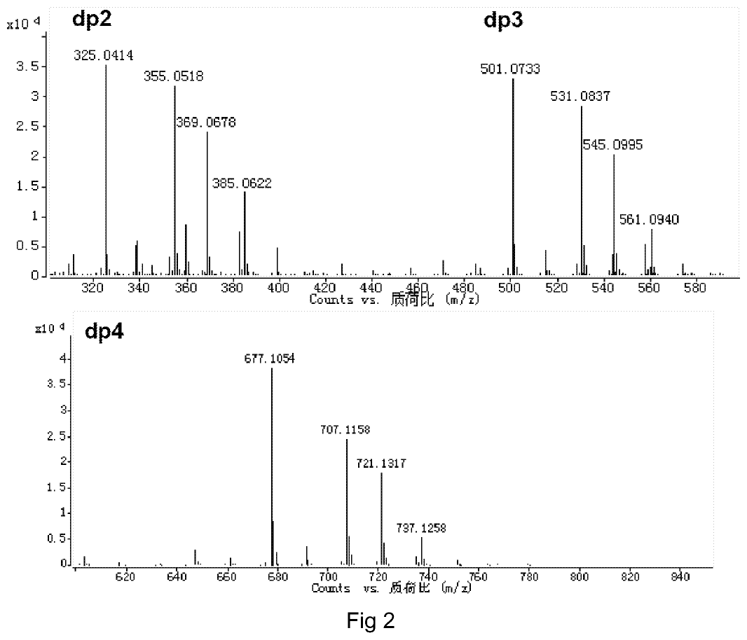

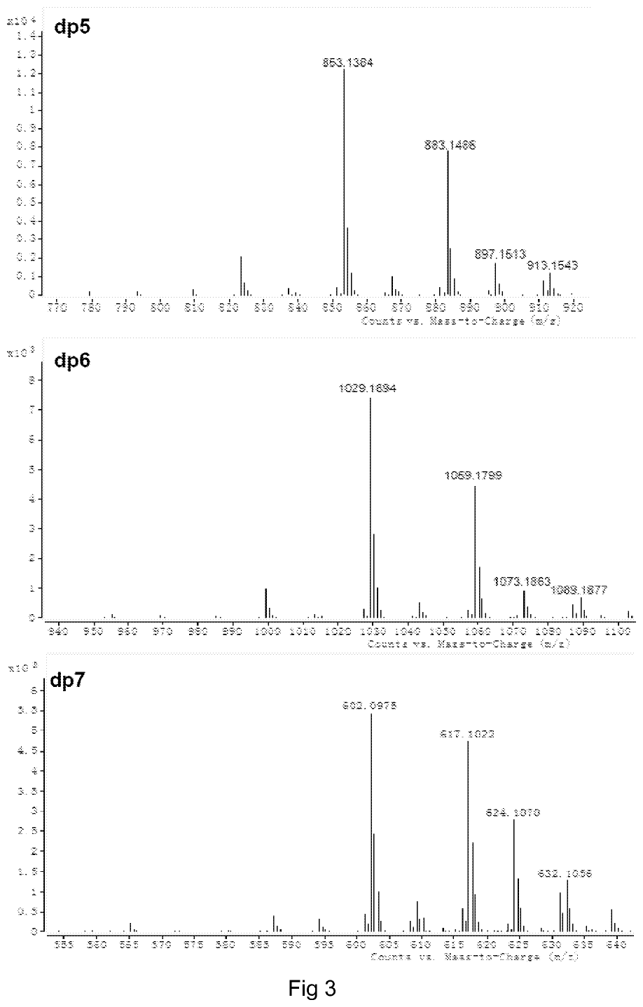

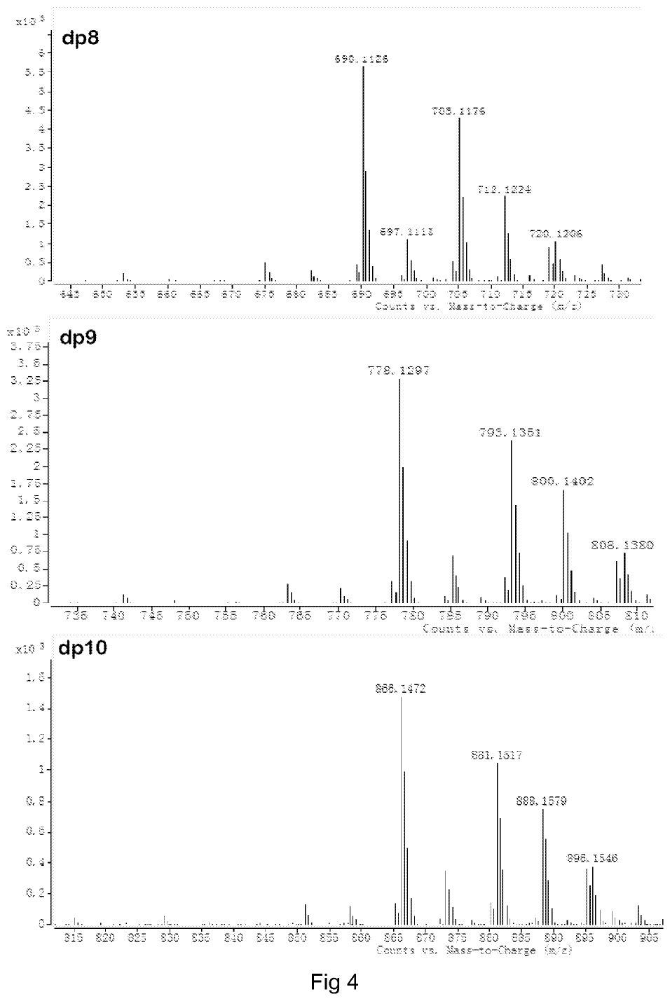

The mass spectra of oligosaccharides with various polymerization degrees are shown in FIGS. 2-4. Various signal peaks in the mass spectra were assigned, confirming the molecular structure of all oligosaccharides in the raw material, i.e., the structure shown in General Formula (III). See Table 1 below for the signal assignments and the structures corresponding to the signals.

Table 1: six diacid structures in oligosaccharides with different polymerization degrees in the raw material and their mass-to-charge ratios in mass spectra.

| | | Molecular |
|---|---|---|
| No. [symbol] | Molecular Structure [symbol] | Formula [symbol] |
| 1 [symbol] | 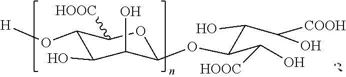 | (C₆H₈O₆)ₙC₆H₁₀O₈ [symbol] n = 1-9 [symbol] |
| 2 [symbol] | 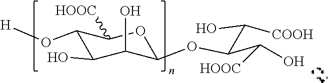 | (C₆H₈O₆)ₙC₅H₈O₇ [symbol] n = 1-9 [symbol] |
| 3 [symbol] | 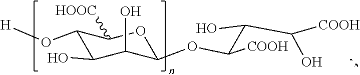 | (C₆H₈O₆)ₙC₅H₈O₇ [symbol] n = 1-9 [symbol] |
| 4 [symbol] | 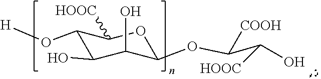 | (C₆H₈O₆)ₙC₄H₆O₅ [symbol] n = 1-9 [symbol] |
| 5 [symbol] | 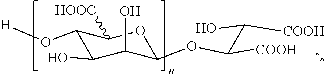 | (C₆H₈O₆)ₙC₄H₆O₅ [symbol] n = 1-9 [symbol] |
| 6 [symbol] | 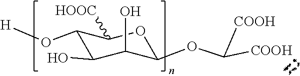 | (C₆H₈O₆)ₙC₃H₄O₅ [symbol] n = 1-9 [symbol] |

| | Mass-to-Charge Ratio (m/z) [symbol] | | | | | | | | |
|---|---|---|---|---|---|---|---|---|---|
| | n = 1 [symbol] | n = 2 [symbol] | n = 3 [symbol] | n = 4 [symbol] | n = 5 [symbol] | n = 6 [symbol] | n = 7 [symbol] | n = 8 [symbol] | n = 9 [symbol] |
| No. [symbol] | [M − 1] [symbol] | [M − 1] [symbol] | [M − 1] [symbol] | [M − 1] [symbol] | [M − 1] [symbol] | [M − 1] [symbol] | [M − 1] [symbol] | [M − 1] [symbol] | [M − 1] [symbol] |
| 1 [symbol] | 385 [symbol] | 561 [symbol] | 737 [symbol] | 913 [symbol] | 1089 [symbol] | 1265 [symbol] | 720 [symbol] | 808 [symbol] | 896 [symbol] |
| 2 [symbol] | 355 [symbol] | 531 [symbol] | 707 [symbol] | 883 [symbol] | 1059 [symbol] | 1235 [symbol] | 705 [symbol] | 793 [symbol] | 881 [symbol] |
| 3 [symbol] | 355 [symbol] | 531 [symbol] | 707 [symbol] | 883 [symbol] | 1059 [symbol] | 1235 [symbol] | 705 [symbol] | 793 [symbol] | 881 [symbol] |
| 4 [symbol] | 325 [symbol] | 501 [symbol] | 677 [symbol] | 853 [symbol] | 1029 [symbol] | 1205 [symbol] | 690 [symbol] | 778 [symbol] | 866 [symbol] |
| 5 [symbol] | 325 [symbol] | 501 [symbol] | 677 [symbol] | 853 [symbol] | 1029 [symbol] | 1205 [symbol] | 690 [symbol] | 778 [symbol] | 866 [symbol] |
| 6 [symbol] | 295 [symbol] | 471 [symbol] | 647 [symbol] | 823 [symbol] | 999 [symbol] | 1175 [symbol] | 675 [symbol] | 763 [symbol] | 851 [symbol] |

[symbol]

indicates data missing or illegible when filed

It was found from the above mass spectrometric structural analysis that the mannuronic acid or the guluronic acid at the reducing end of the sugar chain in the raw material was oxidized to a saccharic acid structure (see General Formula IV for the structure), which could be a mannaric acid or guluronic acid structure comprising 6 carbon atoms (m+m′=3) with a content of about 10%˜30%, or a decarboxylation product of mannaric acid or guluronic acid, i.e., a saccharic diacid comprising 5 carbons (m+m′=2) (30-50%) and a saccharide diacid with 4 carbons (m+m′=1) (30%˜40%).

Step 4) NMR Analysis of Guluronic Acid Content in Alginic Oligosaccharic Diacid Raw Material

Sample preparation: 50 mg of the sample to be tested was weighed, dissolved in 0.5 ml D2O, and lyophilized; 0.5 ml deuterated heavy water was added for dissolution; lyophilization was again performed; finally, the lyophilized sample powder was dissolved with an appropriate amount of heavy water, all of which was transferred to an NMR tube and prepared to a 100 mg/ml solution to be tested; and 0.01% (w/v) deuterated TSP (trimethylsilylpropionic) sodium salt was added as an internal standard.

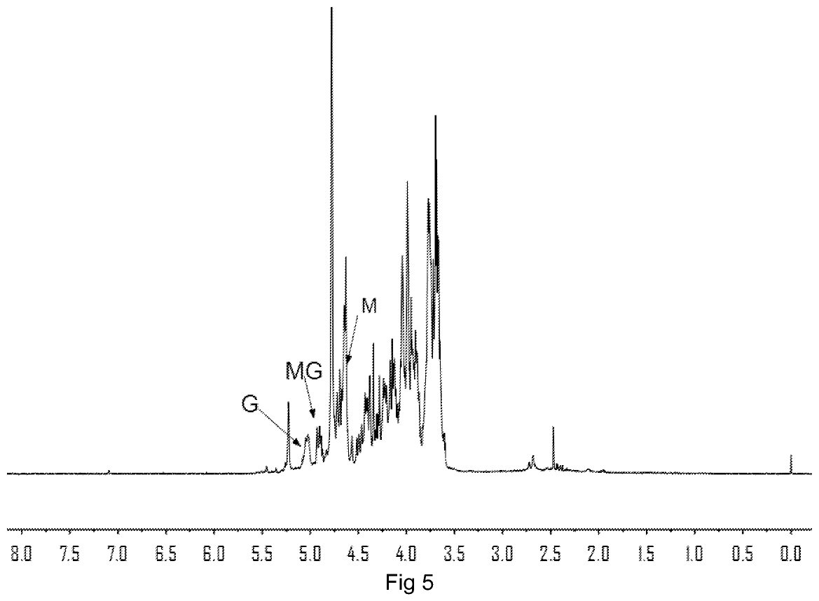

Nuclear magnetic data acquisition and processing: 400M Fourier transform nuclear magnetic resonance instrument collected one-dimensional hydrogen spectrum at room temperature. The pulse sequence was 45° pulses, each acquisition was 4 seconds, the relaxation time was 1 second, and the accumulation was 20 times, and the spectral width was from −2 ppm to 10 ppm. After data collection, Fourier transform was used to obtain a one-dimensional hydrogen spectrum, and the TSP methyl hydrogen signal was set to 0.00 ppm. The proton nuclear magnetic resonance spectrum of AI is shown in FIG. 5. In FIG. 5, the multiplet with a chemical shift of 4.6 ppm is the hydrogen signal at C-1 position of mannuronic acid (M), 5.0 ppm is the hydrogen signal at C-1 position of guluronic acid (G), 4.9 ppm is the C-1 hydrogen signal of chimeric segment of mannuronic acid and guluronic acid (MG). The formula for calculating the content of guluronic acid is:

$$G \% = \frac{{I}_{5.} + 0.5 {I}_{4.9}}{{I}_{5.} + {I}_{4.6} + {I}_{4.9}} ⨯ 100 \%$$

In the above formula, I_{4.6}, I_{5.0} and I_{4.9} are respectively the hydrogen signal integral values at the C-1 positions of the mannuronic acid (M), the guluronic acid (G), and the chimeric segment of mannuronic acid and guluronic acid chimeric (MG). By calculation, the content of the guluronic acid in the API is 30%.

### Example 2: Hygroscopic of Alginate Oligosaccharide Diacid API

The alginate oligosaccharide diacid API (purity 99.2%) prepared in example 1 changed from a uniformly dispersed light yellow fine powder state to yellow brown paste after being placed for 5 days in a high humidity (RH 75%, 25° C.), and the surface was smooth and glossy; it was weighed, and found to have gained more than 30% by weight.

### Example 3: Formulation Examples

150 g of the alginate oligosaccharide diacid API (particle size detection: 100% passed through a 60 mesh sieve, and 80% passed through a 100 mesh sieve) prepared according to the example 1 and 55 g of corn starch (commercial product, which meets the standard of Chinese pharmacopoeia, and the particle size: all pass through a 100 mesh sieve) were mixed uniformly, 85% of ethanol was used as a binder to prepare soft materials, a sieve with the aperture of 4±0.5 mm was used for granulation, dried at 60-70° C., the sieve with the aperture of 1.2±0.2 mm was used for finishing granules, 2 g of magnesium stearate and 2 g of talcum powder were added, the mixture was uniformly mixed and filled into capsules, and about 1000 granules were obtained.

According to the above detection method, the dissolution rate of the main active ingredients was 91.6% in 30 minutes, the content of formic acid was 0.037%, the weight average molecular weight was 925.1 Da, and the molecular weight distribution was 1.051.

### Example 4

150 g of the alginate oligosaccharide diacid API (particle size detection: 100% passed through a 60 mesh sieve, and 80% pass through a 100 mesh sieve) prepared according to the example 1 and 65 g of corn starch were mixed uniformly, 85% of ethanol was used as a binder to prepare soft materials, a sieve with the aperture of 4±0.5 mm was used for granulation, dried at 60-70° C., the sieve with the aperture of 1.2±0.2 mm was used for finishing granules, 3 g of magnesium stearate and 3 g of talcum powder were added, the mixture was uniformly mixed and filled into capsules, and about 1000 granules were obtained. According to the above detection methods, the dissolution rate of the main active ingredients was 93.8% in 30 minutes, the content of formic acid was 0.041%, the weight average molecular weight was 907.9 Da, and the molecular weight distribution was 1.048.

### Example 5

450 g of the alginate oligosaccharide diacid API (particle size detection: 100% passed through a 60 mesh sieve, and 85% pass through a 100 mesh sieve) prepared according to the example 1 and 65 g of corn starch were mixed uniformly, 85% of ethanol was used as a binder to prepare soft materials, a sieve with the aperture of 4±0.5 mm was used for granulation, dried at 60-70° C., the sieve with the aperture of 1.2±0.2 mm was used for finishing granules, 2.5 g of magnesium stearate and 5 g of talcum powder were added, the mixture was uniformly mixed and filled into capsules, and about 1000 granules were obtained. Detection showed that the dissolution rate of the main active ingredients was 94.6% in 30 minutes, the content of formic acid was 0.039%, the weight average molecular weight was 915.2 Da, and the molecular weight distribution was 1.044.

### Example 6

450 g of the alginate oligosaccharide diacid API (particle size detection: 100% passed through a 60 mesh sieve, and 90% pass through a 100 mesh sieve) prepared according to the example 1 and 45 g of sodium carboxymethylcellulose were mixed uniformly, 85% of ethanol was used as a binder to prepare soft materials, a sieve with the aperture of 4±0.5 mm was used for granulation, dried at 60-70° C., the sieve with the aperture of 1.2±−0.2 mm was used for finishing granules, 2.5 g of magnesium stearate and 5 g of talcum powder were added, the mixture was uniformly mixed and tableted into a thin film coat, about 1000 granules were obtained s. Detection showed that the dissolution rate of the main active ingredients was 93.8% in 30 minutes, the content of formic acid was 0.037%, the weight average molecular weight was 918.9 Da, and the molecular weight distribution was 1.046.

### Example 7

450 g of the alginate oligosaccharide diacid API prepared according to example 1 (particle size detection: 100% passed through a 60 mesh sieve, and 90% passed through a 100 mesh sieve) and 85 g of corn starch were mixed uniformly, boiled and granulated with 5% of corn starch slurry, and a sieve with the aperture of 1.2±0.2 mm was used for finishing granules, then added 2.5 g of magnesium stearate and 5 g of talcum powder, mixed uniformly, and tableted into thin film coat, about 1000 granules were obtained. Detection showed that dissolution rate of the main active ingredients was 95.1% in 30 minutes, the content of formic acid was 0.032%, the weight average molecular weight was 919.3 Da, and the molecular weight distribution was 1.049.

### Example 8

150 g of the alginate oligosaccharide diacid API prepared according to example 1 (particle size detection: 100% passed through a 60 mesh sieve, and 90% passed through a 100 mesh sieve) and 75 g of microcrystalline cellulose were mixed uniformly, 85% ethanol was used as a binder to prepare a soft material, a sieve with the aperture of 4±0.5 mm was used for granulation, dried at 60-70° C., a sieve with the aperture of 1.2±0.2 mm was used for finishing granules, 3 g of magnesium stearate and 3 g of talcum powder were added, the mixture was mixed uniformly, and the mixture was filled into capsules, about 1000 granules were obtained. Detection showed that the dissolution rate of the main active ingredients was 93.8% in 30 minutes, the content of formic acid was 0.041%, the weight average molecular weight was 907.9 Da, and the molecular weight distribution was 1.048.

### Comparative Example

450 g of the alginate oligosaccharide diacid API (the particle size detection: 100% of the alginate oligosaccharide diacid raw material was sieved by a 60 mesh sieve, 30% of the alginate oligosaccharide diacid raw material was sieved by a 100 mesh sieve) and 20 g of corn starch were mixed uniformly, 85% of ethanol was used as a binder to prepare a soft material, a sieve with the aperture of 4±0.5 mm was used for granulating, dried at 80-90° C., the sieve with the aperture of 1.2±0.2 mm was used for finishing granules, 10 g of magnesium stearate and 5 g of talcum powder were added, mixed uniformly, and tableted into thin film coat, about 1000 granules were obtained. Detection showed that the dissolution rate of the main active ingredients was 62.5% in 30 minutes, the content of formic acid was 0.27%, the weight average molecular weight was 905.4 Da, and the molecular weight distribution was 1.046.

### Example 8: Accelerated Stability Test

The compositions prepared in examples 3-8 and comparative examples were subject to accelerated test under 40° C.±2° C./75% RH±5% RH are given in the following table:

| | | time (month) | | | | |
|---|---|---|---|---|---|---|
| sample | Investigation items | 0 | 1 | 2 | 3 | 6 |
| Example 3 | Appearance is | fit | fit | fit | fit | fit |
| | white to light | | | | | |
| | yellow powder or | | | | | |
| | granules | | | | | |
| | Dissolution in 30 | 91.6 | 91.3 | 91.5 | 91.2 | 91.3 |
| | minutes (%) | | | | | |
| | Formic acid (%) | 0.037 | 0.037 | 0.038 | 0.039 | 0.039 |
| | Weight average | 925.1 | 920.1 | 929.5 | 930.6 | 921.4 |
| | molecular weight | | | | | |
| | (Da) | | | | | |
| | Molecular weight | 1.051 | 1.048 | 1.052 | 1.048 | 1.047 |
| | distribution | | | | | |
| Example 4 | Appearance is | fit | fit | fit | fit | fit |
| | white to light | | | | | |
| | yellow powder or | | | | | |
| | granules | | | | | |
| | Dissolution in 30 | 93.8 | 93.6 | 93.1 | 94 | 93.6 |
| | minutes (%) | | | | | |
| | Formic acid (%) | 0.041 | 0.040 | 0.041 | 0.041 | 0.042 |
| | Weight average | 907.9 | 910.1 | 911.9 | 900.2 | 902.4 |
| | molecular weight | | | | | |
| | (Da) | | | | | |
| | Molecular weight | 1.048 | 1.049 | 1.049 | 1.045 | 1.054 |
| | distribution | | | | | |
| Example 5 | Appearance is | fit | fit | fit | fit | fit |
| | white to light | | | | | |
| | yellow powder or | | | | | |
| | granules | | | | | |
| | Dissolution in 30 | 94.6 | 94.2 | 94.6 | 94.3 | 94.5 |
| | minutes (%) | | | | | |
| | Formic acid (%) | 0.039 | 0.039 | 0.040 | 0.041 | 0.042 |
| | Weight average | 915.2 | 915.6 | 918.1 | 909.7 | 920 |
| | molecular weight | | | | | |
| | (Da) | | | | | |
| | Molecular weight | 1.044 | 1.048 | 1.048 | 1.043 | 1.045 |
| | distribution | | | | | |
| Example 6 | Appearance is | fit | fit | fit | fit | fit |
| | white to light | | | | | |
| | yellow powder or | | | | | |
| | granules | | | | | |
| | Dissolution in 30 | 93.8 | 93.4 | 93.5 | 93.3 | 93.4 |
| | minutes (%) | | | | | |
| | Formic acid (%) | 0.037 | 0.038 | 0.037 | 0.039 | 0.040 |
| | Weight average | 918.9 | 915.6 | 912.6 | 916.3 | 915.6 |
| | molecular weight | | | | | |
| | (Da) | | | | | |
| | Molecular weight | 1.046 | 1.046 | 1.047 | 1.042 | 1.044 |
| | distribution | | | | | |
| Example 7 | Appearance is | fit | fit | fit | fit | fit |
| | white to light | | | | | |
| | yellow powder or | | | | | |
| | granules | | | | | |
| | Dissolution in 30 | 95.1 | 95 | 95.3 | 95.1 | 95.4 |
| | minutes (%) | | | | | |
| | Formic acid (%) | 0.032 | 0.033 | 0.034 | 0.034 | 0.035 |
| | Weight average | 919.3 | 917.6 | 915.3 | 919.2 | 918.4 |
| | molecular weight | | | | | |
| | (Da) | | | | | |
| | Molecular weight | 1.049 | 1.05 | 1.047 | 1.049 | 1.046 |
| | distribution | | | | | |
| Example 8 | Appearance is | fit | fit | fit | fit | fit |
| | white to light | | | | | |
| | yellow powder or | | | | | |
| | granules | | | | | |
| | Dissolution in 30 | 93.8 | 93.6 | 93.6 | 93.7 | 93.6 |
| | minutes (%) | | | | | |
| | Formic acid (%) | 0.041 | 0.041 | 0.042 | 0.043 | 0.043 |
| | Weight average | 907.9 | 908.6 | 908.3 | 909.2 | 908.4 |
| | molecular weight | | | | | |
| | (Da) | | | | | |
| | Molecular weight | 1.048 | 1.047 | 1.049 | 1.048 | 1.049 |
| | distribution | | | | | |
| Comparative | Appearance is | fit | fit | fit | yellow | yellow |
| example | white to light | | | | brown | brown |
| | yellow powder or | | | | paste | paste |
| | granules | | | | | |
| | Dissolution in 30 | 62.5 | 62.4 | 63.1 | 62.3 | 62.5 |
| | minutes (%) | | | | | |
| | Formic acid (%) | 0.27 | 0.29 | 0.30 | 0.32 | 0.37 |
| | Weight average | 905.4 | 874.3 | 867.5 | 813.4 | 799.4 |
| | molecular weight | | | | | |
| | (Da) | | | | | |
| | Molecular weight | 1.046 | 1.186 | 1.25 | 1.32 | 1.57 |
| | distribution | | | | | |

Clinical Study:

The study was a 36 week, randomized, double-blind, placebo-controlled, multicenter clinical trial with 818 patients randomized into the group and 450 mg of alginate oligosacchridic diacid composition was administrated orally twice daily. The subjects were aged 50-85 years, and scored 11-26 on the MMSE scale. MRI showed that the medial temporal atrophy visual rating scale (MTA) was not greater than grade 2, the leukosis Fazekas scale was less than grade 3, no more than 2 infarcts with a diameter greater than 2 cm, no infarcts in critical brain areas, which met the clinical diagnosis criteria of mild to moderate Alzheimer's disease. The primary efficacy index was the inter-group differences compared with the placebo group of the changes from baseline after 36 weeks of treatment in the cognitive function assessment scale ADAS-cog12, the secondary efficacy index was the inter-group differences in changes from baseline in Clinician Interviewed Impression Change Scale CIBIC-plus and Activities of Daily Living ADCS-ADL Scale and Psycho-Behavioural Questionnaire NPI at 36 weeks of treatment. The safety evaluation indexes comprise adverse events, laboratory examinations, vital signs, electrocardiograms, physical examinations etc.

Clinical research results show that the alginate oligosaccharide diacid composition can evidently improve cognitive dysfunction of AD patients. Compared with a placebo group, the average improvement value of the ADAS-cog12 scale is 2.54, which has extremely remarkable statistical significance (p<0.0001). The efficacy was particularly significant in the subgroup with a high disease severity (MMSE scale score of 11-14), with an average improvement on the ADAS-cog12 scale of 4.55 compared to the placebo group. The alginate oligosaccharide diacid composition has a remarkable improvement trend (p is 0.059) on a secondary curative effect index CIBIC-plus; however, as for the ADCS-ADL tables and the NPI questionnaire, no statistical significance was observed. Compared with the placebo group, the alginate oligosaccharide diacid composition group has no significant statistical difference in the incidence of adverse events or serious adverse events, and has good safety and strong tolerance.

1. A pharmaceutical composition, comprising 40-90 wt % alginate oligosaccharide diacid with structure shown in formula (IV) or a pharmaceutically acceptable salt thereof, and 8-50 wt % filler; wherein the filler is selected from starch and/or cellulose; 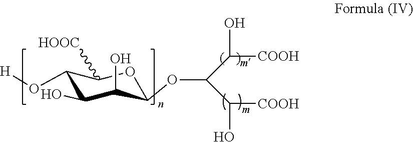
wherein n is an integer selected from 1 to 9, m is selected from 0, 1 or 2, and m′ is selected from 0 or 1.

2. The pharmaceutical composition of claim 1, wherein the filler is at least one selected from the group consisting of dry starch, corn starch, pregelatinized starch, microcrystalline cellulose, sodium carboxymethylcellulose, and croscarmellose sodium.

3. The pharmaceutical composition of claim 1, wherein the particle size of said alginate oligosaccharide diacid or a pharmaceutically acceptable salt thereof is controlled such that 100% pass through a 60 mesh sieve, and more than 70% pass through a 100 mesh sieve.

4. The pharmaceutical composition of claim 1, further comprising 0 to 10 wt % of binder, and 0.1 to 3 wt % of lubricant; wherein the binder is at least one of starch, sodium carboxymethyl cellulose, hydroxypropyl methyl cellulose and polyvinylpyrrolidone; and the lubricant is at least one selected from talcum powder, magnesium stearate, silica gel micropowder and hydrogenated vegetable oil.

5. The pharmaceutical composition according to claim 1, wherein the total weight of the alginate oligosacchridic acids wherein n=1-5 or a pharmaceutically acceptable salt thereof is more than 60% of the total weight of the alginate oligosacchridic acids or a pharmaceutically acceptable salt thereof; and the total weight of guluronic acid diacid accounts for 0-50% of the total weight of alginate oligosaccharide diacid or a pharmaceutically acceptable salt thereof.

6. The pharmaceutical composition according to claim 1, wherein the total weight of mannuronic acid and/or guluronic acid wherein n=1-3 in the alginate oligosaccharide diacid accounts for 20-70% of the total weight of the alginate oligosaccharide diacid or a pharmaceutically acceptable salt thereof.

7. The pharmaceutical composition according to claim 6, wherein the total weight of the guluronic acid is between 5 and 50% of the total weight of the alginate oligosaccharide diacid or a pharmaceutically acceptable salt thereof.

8. The pharmaceutical composition of claim 7, wherein the weight of the alginate oligosaccharide diacid or a pharmaceutically acceptable salt thereof with each polymerization degree based on the total weight of the alginate oligosaccharide diacid or a pharmaceutically acceptable salt thereof is as follows: 5-30% of disaccharide, 15-35% of trisaccharide, 15-35% of tetrasaccharide and 20-45% of total content of pentasaccharide, hexasaccharide, heptasaccharide, octasaccharide, nonasaccharide and decasaccharide.

9. The pharmaceutical composition of claim 6, wherein the weight of the alginate oligosaccharide diacid or a pharmaceutically acceptable salt thereof with each polymerization degree accounts for the following percentage of the total weight of the alginate oligosaccharide diacid or a pharmaceutically acceptable salt thereof: 5-30% of disaccharide, 15-35% of trisaccharide, 15-35% of tetrasaccharide, 10-25% of pentasaccharide, 5-15% of hexasaccharide, 3-10% of heptasaccharide, 2-5% of octasaccharide, 1-5% of nonasaccharide and 1-5% of decasaccharide.

10. The pharmaceutical composition of claim 9, wherein the weight of the alginate oligosaccharide diacid or a pharmaceutically acceptable salt thereof with each polymerization degree based on the total weight of the alginate oligosaccharide diacid or a pharmaceutically acceptable salt thereof is as follows: 10-29% of disaccharide, 18-32% of trisaccharide, 15-30% of tetrasaccharide, 15-20% of pentasaccharide, 5-10% of hexasaccharide, 3-5% of heptasaccharide, 2-3% of octasaccharide, 1-3% of nonasaccharide and 1-3% of decasaccharide.

11. The pharmaceutical composition according to claim 1, wherein the content of formic acid or a salt thereof is lower than 0.2%.

12. The pharmaceutical composition of claim 1, wherein the pharmaceutically acceptable salt is a sodium or potassium salt.

13. (canceled)

14. A method of treating a patient having a disease selected from the group consisting of Alzheimer's disease, Parkinson's disease, inflammation, pain, diabetes or vascular dementia, comprising administering to a subject in need thereof a pharmaceutical composition according to claim 1 in unit dosage form.

15. A pharmaceutical formulation comprising a pharmaceutical composition according to claim 1.

16. The pharmaceutical formulation of claim 15, wherein the formulation is administered orally.

17. The pharmaceutical formulation of claim 15, wherein the formulation is a tablet, a granule, or a capsule.

18. A process for preparing a pharmaceutical composition according to claim 1, comprising the steps of:
(1) mixing 40-90 wt % of alginate oligosaccharide diacid or a pharmaceutically acceptable salt thereof and 8-50 wt % of filler uniformly; and
(2) granulating the uniformly mixed materials to obtain the pharmaceutical composition; or directly tabletting the uniformly mixed material obtained in the step (1) to obtain the pharmaceutical composition,
wherein a binder is optionally added in step (1) or step (2) in an amount of 0 to 10% by weight based on the mixture obtained in step (1).

19. The method of claim 18, wherein the granulation in step (2) is dry granulation, wet granulation or boiling granulation.

20. The method of claim 19, wherein 0.1-3 wt % of lubricant is further added after the completion of the granulation in step (2).

21. The method according to claim 18, wherein step (2) is followed by step (3): coating the plain tablets obtained by tabletting in the step (2) to obtain the pharmaceutical composition.

22. The method of claim 18, comprising the steps of:
(1) mixing 40-90 wt % of alginate oligosaccharide diacid or a pharmaceutically acceptable salt thereof and 8-50 wt % of filler uniformly;
(2) adding binder into the mixed materials to make soft materials, granulating with 4±0.5 mm mesh sieve, drying at 60-70° C., finishing granules with 1.2±0.2 mm mesh sieve, adding lubricant, mixing uniformly, and filling into capsule or tableting to thin film coat.
